(19) United States
(12) Patent Application Publication
(10) Pub. No.: US 2018/0103906 A1
(43) Pub. Date: Apr. 19, 2018

(54) MULTIFUNCTION BUCKLE FOR A WEARABLE DEVICE

(71) Applicant: CU Wellness, Inc., Los Altos Hills, CA (US)

(72) Inventors: Shripal Gandhi, Pune (IN); Keyur Patel, Mountain View, CA (US); Anal Patel, Mountain View, CA (US); Susanto Purnama, Mountain View, CA (US)

(21) Appl. No.: 15/725,837

(22) Filed: Oct. 5, 2017

Related U.S. Application Data
(60) Provisional application No. 62/409,250, filed on Oct. 17, 2016.

(51) Int. Cl. A61B 5/00; A44B 11/00; A61B 5/11; A61B 5/0205; A61B 5/16

(52) U.S. Cl. CPC A61B 5/6831; A61B 5/02438; A61B 5/1118; A61B 5/112; A61B 5/4809; A61B 5/0077; A61B 5/1128; A61B 5/7282; A61B 5/002; A61B 5/0024; A61B 5/02055; A61B 5/165; A61B 5/4266; A61B 5/7445; A61B 5/7455; A61B 2560/0475; A61B 2562/0257; A44B 11/005

(57) ABSTRACT

The present disclosure relates to the field of electronic engineering. The present disclosure envisages a multifunction buckle that integrates multiple health monitoring devices. The multifunction buckle comprises a health sensing module, an activity tracking module, a signal conditioning unit, a processing unit, a notification module, and a communication module. The health sensing module has a plurality of health sensors configured to sense a plurality of health parameters associated with a user. The activity tracking module comprises a pedometer, a sleep detection module, and a gesture detection module. The signal conditioning unit co-operates with the health sensing module and the activity tracking module. The processing unit co-operates with the signal conditioning unit, the health sensing module and the activity tracking module. The notification module co-operates with the processing unit and notifies the user. The communication module receives at least one communication signal from the processor and enables bi-directional communication with communicatively coupled device.

### FIELD

The present disclosure relates to the field of electronics engineering. Particularly, the present disclosure relates to the buckles having sensors.

### BACKGROUND

Conventionally, people who monitor their health conditions, frequently visit health care centers in order to perform routine tests for measuring biometric parameters such as body temperature, heart rate, blood pressure, ECG (Electrocardiography), glucose level, and body fat analysis. Frequent trips to the health care centre are not only inconvenient for the people but are also costly and time consuming. Further, to avoid frequent trips to the health care centre, a caretaker is usually hired for performing such routine tests which also turn out to be expensive and may the caretaker may not be able to provide a quality service. Additionally, an area of concern is the requirement for the health care centers to provide a quick response to emergency cases such as falling of a patient, irregular changes in the heartbeat rate, change in ECG, and no movement condition of a patient under observation.

Furthermore, a user typically has to use multiple health monitoring devices such as a blood pressure monitoring device, an ECG machine, and a pedometer. Conventionally, since the multiple health monitoring devices used for measuring biometric parameters are not integrated, therefore completely distributed records are generated which are difficult to maintain and track. Further, the use of multiple health monitoring devices having different power handling capabilities results in high power dissipation in totality. In addition, the multiple electronic devices occupy a considerable amount of space which is not desired during travel.

Conventionally, various wearable devices such as wristwatches, bands, and bracelets have been developed with an added functionality of measuring biometric parameters such as body temperature, heart rate, blood pressure, ECG (Electrocardiography), glucose level, and body fat analysis associated with the user, separately. These dual function wearable devices are a lot more complex and expensive than the conventional wearable devices. Further, for example, the primary function of the smart watch is to display time and if the primary function of the smart wristwatch fails, then the smart wristwatch is either repaired or replaced which increases the cost.

Therefore, there is felt a need of a multifunction buckle that can be retrofittedly attached to the conventional wearable devices and alleviates or eliminates the above-mentioned drawbacks.

### OBJECTS

Some of the objects of the present disclosure, which at least one embodiment herein satisfies, are as follows.

It is an object of the present disclosure to ameliorate one or more problems of the prior art or to at least provide a useful alternative.

An object of the present disclosure is to provide a multifunction buckle that is compact.

Another object of the present disclosure is to provide a multifunction buckle that requires less power.

Still another object of the present disclosure is to provide a multifunction buckle that can be retrofitted on different wearable devices.

Yet another object of the present disclosure is to provide a multifunction buckle that instantly transfers the measured health parameters to a concerned authority or health care centre.

Other objects and advantages of the present disclosure will be more apparent from the following description, which is not intended to limit the scope of the present disclosure.

### BRIEF DESCRIPTION OF ACCOMPANYING DRAWING

A multifunction buckle for a wearable device of the present disclosure will now be described with the help of the accompanying drawings, in which:

FIG. 1 illustrates a perspective view of a multifunction buckle attached to a wrist watch; and

FIG. 2 illustrates a block diagram of the multifunction buckle of FIG. 1.

FIG. 3 illustrates a flow process for sensing, analyzing, comparing and transmitting information using a multifunction buckle of FIG. 1.

### DETAILED DESCRIPTION

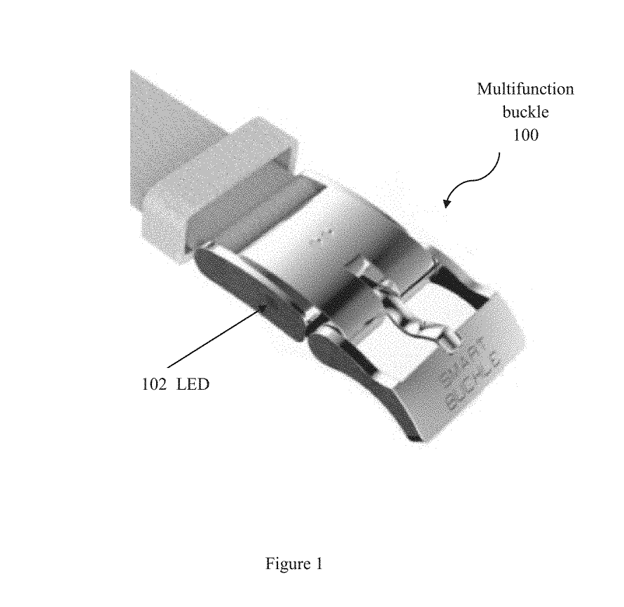

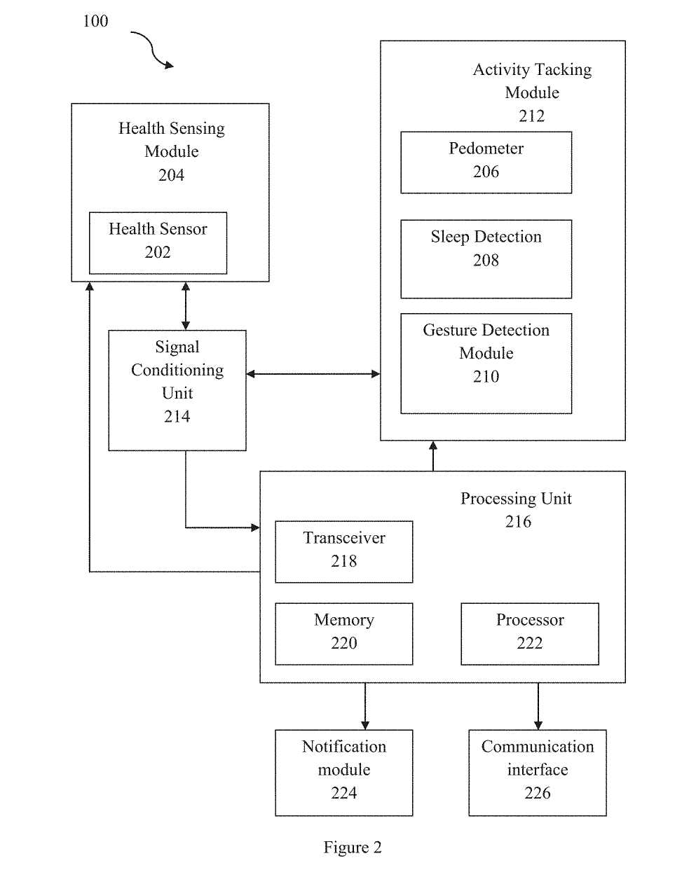

The present disclosure envisages a multifunction buckle for a wearable device. A preferred embodiment of the multifunction buckle, of the present disclosure will now be described in detail with reference to the accompanying drawing. The preferred embodiment does not limit the scope and ambit of the disclosure. The description provided is purely by way of example and illustration. FIG. 1 illustrates a perspective view of a multifunction buckle 100 that is attached to a strap of a wearable device. FIG. 2 illustrates a block diagram of the multifunction buckle 100.

The multifunction buckle 100 for a wearable device comprises a health sensing module 204, an activity tracking module 212, a signal conditioning unit 214, a processing unit 216, a notification module 224, and a communication module 226. In an embodiment, the multifunction buckle 100 further includes at least one battery. The at least one battery (not labelled in figures) provides power to the multifunction buckle 100. In an embodiment, the wearable device may be a wristwatch, a footwear, a cap, a bracelet, a wristband, a waist-belt, and the like.

The health sensing module 204 has a plurality of health sensors 202 configured to sense a plurality of health parameters associated with a user and generate a plurality of health signals. In an embodiment, the plurality of health sensors 202 is selected from the group consisting of calorie sensors, biometric sensors, heart-rate sensors, blood oxygen sensors, sweat sensors, temperature sensors, emotion tracking sensors, stress tracking sensors, blood pressure monitoring sensors, and skin conductance sensors.

The activity tracking module 212 comprises a pedometer 206, a sleep detection module 208, and a gesture detection module 210. The pedometer 206 is configured to detect a physical motion and count steps travelled by users and is further configured to generate at least one pedometer signal. The sleep detection module 208 is configured to generate sleep signals that convey information related to one or more physiological functions that indicate sleep stage of the user. The gesture detection module 210 is configured to detect gestures of the user and generate a plurality of gesture signals. In an embodiment, the gesture detection module 200 includes at least one camera (not shown in figures) and at least one proximity sensor (not shown in figures). In an exemplary embodiment, the gesture detection module 210 may be configured to perform remote operations on at least one communicatively coupled device, such as mobiles, tablets, laptop, personal-digital-assistant (PDA), and the like. In another embodiment, the remote operations may be activating camera of the at least one communicatively coupled device for image capturing, video recording, and the like. In yet another embodiment, the remote operation may be controlling music, video, opening a document, and the like on the at least one communicatively coupled device.

The signal conditioning unit 214 co-operates with the health sensing module 204 and the activity tracking module 212. The signal conditioning unit 214 is configured to receive the plurality of health signals, the at least one pedometer signal, the sleep signals, and the plurality of gesture signals. The signal conditioning unit 214 is further configured to generate a conditioned health data. Additionally, the signal conditioning unit is configured to generate conditioned activity data, wherein the conditioned activity data includes pedometer data, sleep data, and gesture data.

The processing unit 216 co-operates with the signal conditioning unit 214, the health sensing module 204 and the activity tracking module 212. The processing unit 216 comprises a transceiver 218, a memory 220, and a processor 222. The transceiver 218 is configured to receive the plurality of conditioned health data and the conditioned activity data associated with the user when the wearable device is worn by the user. The memory 220 co-operates with the transceiver 218 and is configured to receive and store the plurality of conditioned health data and the conditioned activity data. The memory 220 is further configured to store a pre-determined set of threshold ranges associated with the user. Further, the processor 222 co-operates with the memory 220 and is configured to analyze and compare the plurality of conditioned health data and the conditioned activity data with the pre-determined set of threshold ranges stored in the memory. The processor 222 is further configured to generate at least one information signal for at the notification module and the communication module. In an embodiment, the processing unit 216 may be an application specific integrated circuit (ASIC), a field programmable grid array (FPGA), an arm processor, and the like. In an embodiment, the at least one information signal is selected from the group consisting of notification signal and communication signal. In another embodiment, the processor 222 is further configured to generate a first activation signal for controlling the operation of the health sensing module 204. In still another embodiment, the processor 222 is configured to generate a second activation signal for controlling the operation of the activity tracking module 212.

The notification module 224 co-operates with the processing unit 216. The notification module 224 is configured to receive the at least one information signal and provide notifications to the user based on the at least one information signal. In an embodiment, the notification module 224 includes at least one light emitting diode for visual notifications and at least one motor for providing tactile notifications. In one embodiment, the notification module 224 may provide notifications for events related to the at least one communicatively coupled device, such as mobiles, tablets, and the like. In another embodiment, the at events related to the at least one communicatively coupled device may be messages, calls, emails, and the like. In one embodiment, the communicatively coupled device may be remotely located.

A communication module 226 co-operates with the processing unit 216. The communication module 226 is configured to receive the at least one communication signal. The communication module 226 is further configured to enable a bi-directional communication with the at least one communicatively coupled device based on the at least one communication signal. In one embodiment, the communication module 226 may include a near field communication module (NFC), and a far field communication module.

In an embodiment, the processor 222 may analyze and compare the health data and sleep data with the predetermined set of threshold ranges. If at least one of the health data, and the sleep data is above or below the predetermined set of threshold ranges, then the processing unit 216 may send the at least one notification signal and the at least one communication signal to the notification module 224 and the communication module 226 respectively. The notification module may notify the user by means of the light emitting diode for visual notification and the motor for tactile notification. The communication module 226 instantly transfers the health parameters associated with the user to the communicatively coupled device that may be remotely located. In an exemplary embodiment, the communicatively coupled device may be remotely located or at the user's health care center.

In another embodiment, the processor 222 may analyze and compare the gesture data with the predetermined ranges stored in the memory 220. If the gesture data is within the predetermined set of range, then the processing unit 216 may generate:
a first activation signal and activate the health sensing module 204; and
a second activation signal and activate the activity tracking module 212.

In yet another embodiment, the multifunction buckle 100 may be further configured to perform financial transactions.

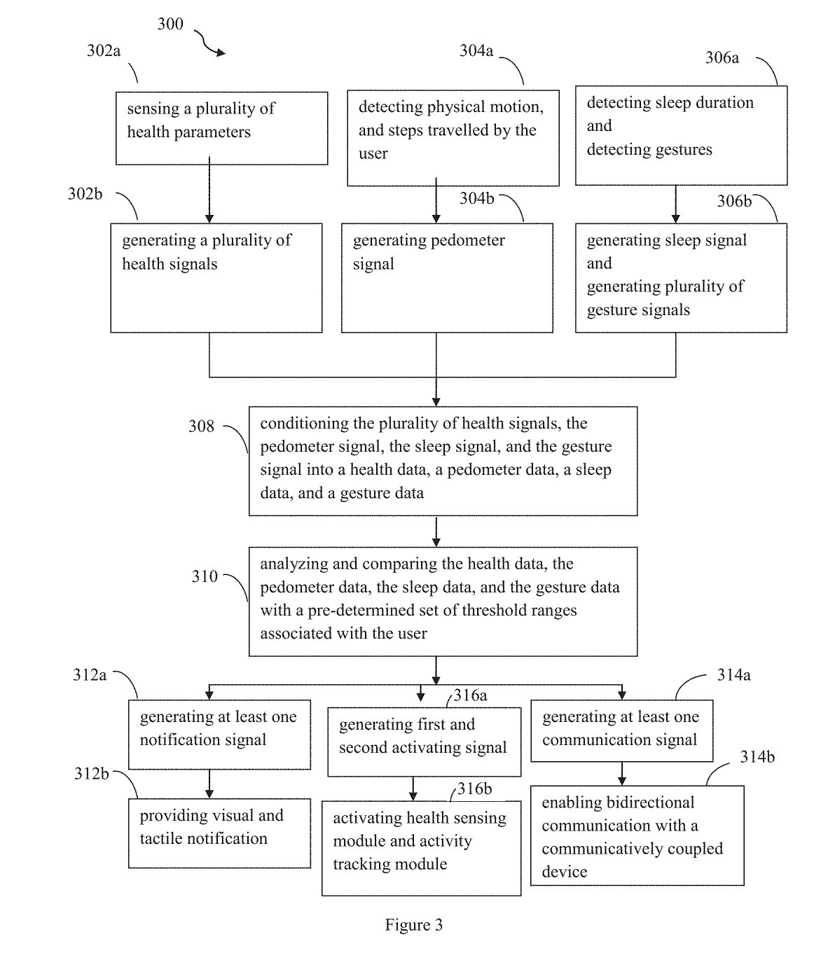

FIG. 3 illustrates a process 300 for sensing, analyzing, comparing and transmitting information using the multifunction buckle 100.

Block 302a: sensing, by the plurality of health sensors 202, the plurality of health parameters associated with a user;

Block 302b: generating, by the plurality of health sensors 202, the plurality of health signals;

Block 304a: detecting, by the pedometer 206, physical motion and steps travelled by the user;

Block 304b: generating, by the pedometer 206, a pedometer signal;

Block 306a: detecting, by the sleep detection module 208 and the gesture detection module 210, sleep duration and gesture of the user;

Block 306b: generating, by the sleep detection module 208 and the gesture detection module 210, sleep signal and gestures signal;

Block 308: conditioning, by the signal conditioning unit 214, the plurality of health signals, the at least one pedometer signal, the sleep signals, and the plurality of gesture signal and generating health data, pedometer data, sleep data, and gesture data;

Block 310: analyzing and comparing, by the processor 222, the health data, the pedometer data, the sleep data, and the gesture data with the pre-determined set of threshold ranges;

Block 312a: generating, by the processor 222, at least one notification signal;

Block 312b: providing, by the notification module 224, visual and tactile notification;

Block 314a: generating, by the processor 222, at least one communication signal;

Block 314b: enabling, by the communication module 226, bidirectional communication with a communicatively coupled device;

Block 316a: generating, by the processor 222, first activation signal and second activation signal;

Block 316b: activating, by the processor 222, the health sensing module 204 and the activity tracking module 212.

### Technical Advances and Economical Significance

The present disclosure described herein above has several technical advantages including, but not limited to, the realization of a multifunction buckle for a wearable device that:
requires less power;
cost effective;
integrates multiple health monitoring devices; and
instantly transfers the health parameters to the health care provider.

The disclosure has been described with reference to the accompanying embodiments which do not limit the scope and ambit of the disclosure. The description provided is purely by way of example and illustration.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully revealed the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A multifunction buckle for a wearable device, said multifunction buckle comprising:
a health sensing module configured to sense a plurality of health parameters and generate a plurality of health signals;
an activity tracking module configured to sense a plurality of activity parameters and generate a plurality of activity signals;
a signal conditioning unit co-operating with said health sensing module and said activity tracking module and configured to generate a plurality of conditioned health data and a plurality of conditioned activity data based on said plurality of health and activity signals respectively;
a processing unit co-operating with said signal conditioning unit to receive said conditioned health data and conditioned activity data, and configured to generate at least one information signal based on said conditioned health data and conditioned activity data; and
a notification module co-operating with said processing unit, and configured to receive said at least one information signal and provide notifications to said user.

2. The multifunction buckle as claimed in claim 1, wherein said activity tracking module includes:
a pedometer configured to detect a physical motion and count steps travelled by said user and further configured to generate at least one pedometer signal;
a sleep detection module configured to generate sleep signals that convey information related to one or more physiological functions that indicate sleep stage of said user; and
a gesture detection module configured to detect gestures of said user and generate a plurality of gesture signals.

3. The multifunction buckle as claimed in claim 1, wherein said plurality of activity signals include the at least one pedometer signal, the sleep signal, and the plurality of gesture signals.

4. The multifunction buckle as claimed in claim 2, wherein said gesture detection module includes at least one camera, and at least one proximity sensor.

5. The multifunction buckle as claimed in claim 2, wherein said signal conditioning unit configured to receive said plurality of health signals, said at least one pedometer signal, said sleep signals, and said plurality of gesture signals and generate a corresponding conditioned health data, and conditioned activity data.

6. The multifunction buckle as claimed in claim 1, wherein said processing unit comprises:
a transceiver configured to receive said plurality of conditioned health data and said plurality of conditioned activity data associated with said user;
a memory configured to cooperate with said transceiver and receive and store said plurality of conditioned health data and said plurality of conditioned activity data, and further configured to store a pre-determined set of threshold ranges associated with said user; and
a processor co-operating with said memory and configured to analyze and compare said plurality of conditioned health data and said plurality of conditioned activity data based on said pre-determined set of threshold ranges and further configured to generate said at least one information signal.

7. The multifunction buckle as claimed in claim 6, wherein said at least one information signal is selected from the group consisting of notification signal and communication signal.

8. The multifunction buckle as claimed in claim 6, wherein said processor is selected from the group consisting of an application specific integration circuit (ASIC), an FPGA (field programmable gate array), an embedded processor, an ARM processor, a PIC controller, and combination thereof

9. The multifunction buckle as claimed in claim 6, wherein said processor further configured to generate a first activation signal for controlling the operation of said health sensing module.

10. The multifunction buckle as claimed in claim 6, wherein said processor further configured to generate a second activation signal for controlling the operation of said activity tracking module.

11. The multifunction buckle as claimed in claim 1, wherein said multifunction buckle further includes a communication module cooperating with said processing unit configured to receive said at least one information signal and further configured to enable a bidirectional communication with at least one communicatively coupled device based on said at least one information signal.

12. The multifunction buckle as claimed in claim 11, wherein said communication module includes a near field communication module (NFC), and a far field communication module.

13. The multifunction buckle as claimed in claim 1, wherein said plurality of health sensors is selected from the group consisting of calorie sensors, biometric sensors, heart-rate sensors, blood oxygen sensors, sweat sensors, temperature sensors, emotion tracking sensors, stress tracking sensors, blood pressure monitoring sensors, humidity sensor, and skin conductance sensors.

14. The multifunction buckle as claimed in claim 1, wherein said notification module includes at least one light emitting diode for visual notification, and at least one motor for providing tactile notification.

15. The multifunction buckle as claimed in claim 1, wherein said multifunction buckle further includes at least one battery for providing power to said multifunction buckle.
